(19) United States
(12) Patent Application Publication
(10) Pub. No.: US 2007/0248960 A1
(43) Pub. Date: Oct. 25, 2007

(54) Arrays containing cleavable RNAi molecules

(76) Inventor: Dianne M. Rees, Sunnyvale, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/407,809

(22) Filed: Apr. 19, 2006

(51) Int. Cl. C12Q 1/68; C12M 3/00

(52) U.S. Cl. 435/6; 435/287.2

(57) ABSTRACT

A nucleic acid array is provided, as well as methods of using the same. In certain embodiments, the nucleic acid array comprises: a) a substrate; and b) an array of features on a surface of the substrate, where the features comprise interfering RNA molecules that are linked to the surface of the substrate by a cleavable linker.

### BACKGROUND

Double-stranded RNA induces potent and specific gene silencing through a process referred to as RNA interference (RNAi). RNAi is mediated by RNA-induced silencing complex (RISC), a sequence-specific, multicomponent nuclease that destroys messenger RNAs homologous to the silencing trigger. RISC is known to contain short RNAs (of approximately 22 nucleotides) derived from the double-stranded RNA trigger. For a review of the RNAi process, see Downward (Brit. Med. J. 2004 328:1245-1248).

### SUMMARY

A nucleic acid array is provided, as well as methods of using the same. In certain embodiments, the nucleic acid array comprises: a) a substrate; and b) an array of features on a surface of the substrate, where the features comprise interfering RNA molecules that are linked to the surface of the substrate by a cleavable linker.

In certain embodiments, the interfering RNA molecules may comprise short interfering RNA molecules or, in other embodiments, may comprise short hairpin interfering RNA molecules.

In certain embodiments, the interfering RNA molecules are linked to the surface via a photocleavable linker or, in other embodiments, a chemically-cleavable linker.

The interfering RNA molecules may be non-covalently or covalently linked to the surface of the substrate.

Also provided is a method that includes: a) contacting a subject nucleic acid array with: i) cells, and ii) an agent that cleaves the cleavable linker to release the interfering RNA molecules from the substrate; b) introducing said interfering RNA molecules into the cells; and c) observing the cells. The agent may be light or a compound, for example.

The cells and the agent may be contacted with the array simultaneously or in series, for example.

The cells may be observed by, e.g., observing a phenotype of the cells, detecting a reporter protein produced by the cells. The cells may be compared to control cells.

In certain embodiments, contacting includes operably engaging the nucleic acid array with a multi-well plate comprising the cells.

Also provided is a system comprising: a subject array and a multi-well plate of cells; wherein the array and multiwell plate are adapted for engaging to each other such that the cells come into contact with one or more pre-determined features of the array.

In certain embodiments, the array and said multi-well plate comprise alignment elements that provide alignment of the array and the multi-well plate as they are being engaged. The alignment elements may include reference marks.

In certain embodiments, the array and the multi-well plate, once engaged, may produce a plurality of sealed chambers.

Also provided is a kit comprising a subject array, a multi-well plate, and a transfection reagent, wherein the array and multiwell plate are adapted for engaging to each other such that the array and the multiwell plate, when engaged, form a plurality of sealed reaction chambers.

### Definitions

The term “nucleic acid” and “polynucleotide” are used interchangeably herein to describe a polymer of any length, e.g., greater than about 10 bases, greater than about 100 bases, greater than about 500 bases, greater than 1000 bases, usually up to about 10,000 or more bases composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes a single stranded multimer of nucleotide of from about 10 to 200 nucleotides. Oligonucleotides are usually synthetic and, in many embodiments, are under 80 nucleotides in length. Oligonucleotides may contain ribonucleotide monomers (i.e., may be oligoribonucleotides) or deoxyribonucleotide monomers.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids that are C-glycosides of a purine or pyrimidine base, polypeptides (proteins), polysaccharides (starches, or polysugars), and other chemical entities that contain repeating units of like chemical structure.

The term “sample” as used herein relates to a material or mixture of materials, typically, although not necessarily, in fluid form, containing one or more components of interest.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “surface-bound nucleic acid”, e.g., a surface bound interfering RNA molecule, refers to a nucleic acid that is immobilized on a surface of a solid substrate, where the substrate can have a variety of configurations, e.g., a sheet, bead, or other structure. In certain embodiments, the nucleic acid probes employed herein are present on a surface of the same planar support, e.g., in the form of an array.

An “array,” includes any two-dimensional or substantially two-dimensional (as well as a three-dimensional) arrangement of spatially addressable regions bearing nucleic acids, particularly oligonucleotides or synthetic mimetics thereof, and the like, e.g., RNAi oligonucleotides. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a surface of the substrate. Depending upon the use, any or all of the arrays may be the same or different from one another and each may contain multiple spots or features. An array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 mm and less than 1.5 mm, such as more than about 0.8 mm and less than about 1.2 mm.

Arrays can be fabricated using drop deposition from pulse-jets of either precursor units (such as nucleotide or amino acid monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotide sequences) such that a region (i.e., a “feature” or “spot” of the array) at a particular predetermined location (i.e., an “address”) on the array contains a particular sequence. Array features are typically, but need not be, separated by intervening spaces.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not spatially distinct. In other words, a mixture is not addressable. To be specific, an array of surface-bound oligonucleotides, as described below, is not a mixture of surface-bound oligonucleotides because the species of surface-bound oligonucleotides are spatially distinct and the array is addressable.

“Isolated” or “purified” generally refers to isolation of a substance (compound, polynucleotide, protein, polypeptide, polypeptide composition) such that the substance comprises a significant percent (e.g., greater than 1%, greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample, that is not found naturally.

The terms “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

If an interfering RNA molecule “corresponds to” or is “for” a certain gene or product thereof, the interfering RNA molecule base pairs with, i.e., specifically hybridizes to, and inactivates that gene or product thereof. As will be discussed in greater detail below, an interfering RNA molecule and the gene or product thereof for that interfering RNA molecule, or complement thereof, usually contain at least one region of contiguous nucleotides that is identical in sequence.

The term “loss-of-function”, as it refers to genes inhibited by the subject RNAi method, refers to a diminishment in the level of expression of a gene (e.g., reducing expression of a gene) when compared to the level in the absence of the RNAi molecule, i.e., in a cell not transfected by the RNAi molecule. By reducing expression is meant that the level of expression of a target gene or coding sequence is reduced or inhibited by at least about 2-fold, e.g., by at least about 5-fold, e.g., 10-fold, 15-fold, 20-fold, 50-fold, 100-fold or more, as compared to a control. By modulating expression of a target gene is meant altering, e.g., reducing, transcription/translation of a coding sequence, e.g., genomic DNA, mRNA etc., into a polypeptide, e.g., protein, product.

The term “expression” with respect to a gene sequence refers to transcription of the gene and, as appropriate, translation of the resulting mRNA transcript to a protein. Thus, as will be clear from the context, expression of a protein coding sequence results from transcription and translation of the coding sequence.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, the terms “transduction” and “transfection” are art recognized and mean the introduction of a nucleic acid, e.g., an RNAi molecule, into a recipient cell by nucleic acid-mediated gene transfer.

As used herein, a “reporter gene construct” is a nucleic acid that includes a “reporter gene” operatively linked to at least one transcriptional regulatory sequence. Transcription of the reporter gene is controlled by these sequences to which they are linked. The activity of at least one or more of these control sequences can be directly or indirectly regulated by the target receptor protein. Exemplary transcriptional control sequences are promoter sequences. A reporter gene is meant to include a promoter-reporter gene construct that is heterologously expressed in a cell.

“Inhibition of gene expression” refers to the absence (or observable decrease) in the level of protein and/or mRNA product from a target gene. “Specificity” refers to the ability of a particular RNAi molecule to inhibit expression of a target gene without directly effecting expression of other genes. The specificity of a particular RNAi molecule can be confirmed by observing an absence of an effect when a control RNAi molecule, e.g., an RNAi molecule having a sequence that is different to the particular RNAi molecule, is transfected into the cell. The consequences of inhibition can, in certain cases, be observed by observing a phenotype of a cell, e.g., an optically detectable phenotype. A phenotype may be observed using, e.g., microcscopy, fluorescence detection, RNA hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line, gene expression can be conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. Lower doses of administered active agent and longer times after administration of active agent may result in inhibition in a smaller fraction of cells (e.g., at least 10%, 20%, 50%, 75%, 90%, or 95% of targeted cells). Quantitation of gene expression in a cell may show similar amounts of inhibition at the level of accumulation of target mRNA or translation of target protein. As an example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

A “cleavably-bound” RNAi molecule or an interfering RNA molecule is linked to a surface by a “cleavable linker” is a substrate-bound RNAi molecule that is releasable from the substrate to which it is bound by exposing the bound molecule to a cleavage-inducing agent, e.g., a chemical or light. The RNAi molecule may be covalently or non-covalently linked to a surface, and cleavage of a cleavable linker results in breaking of a covalent bond that releases an RNIi molecule. Release of a cleavably-bound RNAi is controllable and stimulated by an agent. RNAi molecules that are located proximally to a substrate (or trapped near a substrate) but diffusible therefrom upon addition of an aqueous solution, e.g., water or cell growth medium are not cleavably-bound to the substrate.

### DESCRIPTION OF THE SPECIFIC EMBODIMENTS

A nucleic acid array is provided, as well as methods of using the same. In certain embodiments, the nucleic acid array comprises: a) a substrate; and b) an array of features on a surface of the substrate, where the features comprise interfering RNA molecules that are linked to the surface of the substrate by a cleavable linker.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Arrays

An array of features comprise interfering RNA molecules (i.e., “RNAi molecules”) is provided, where an interfering RNA molecule is designed to silence expression of a specific gene when introduced into a mammalian cell. The interfering RNA molecules are linked to a surface of the array substrate by a cleavable linker such that the interfering RNA molecule may be released from the substrate by exposing the bound molecule to a cleavage-inducing stimulus, e.g., light. After cleavage, the RNAi molecules can be introduced into cells to effect gene silencing.

In certain embodiments, the RNAi molecules may be, for example, short hairpin inhibitory RNA (shRNA) molecules (Paddison Genes Dev. 2002 16:948-58), which are a substrate for the nuclease Dicer and contain complementary target sequences of 19 to 29 nucleotides (i.e., sense and antisense copies of a target sequence of about 20-25 nucleotides in length) separated by a small linker of 3-8 nucleotides. In other embodiments, the RNAi molecules may be, for example, either or both strands of short inhibitory RNA (siRNA) molecules (Elbashir 2001 Nature 411: 494-498), which are double stranded RNA molecules of 19 to 29 nucleotides (e.g., 20-25 nucleotides). shRNA and siRNA molecules silence gene expression via an RISC-mediated pathway. Further details of such molecules are found in Downward (Brit. Med. J. 2004 328:1245-1248); Zamore et al (Cell 2000 101: 25-33); Matzke (Nat. Rev. Genet. 2005 6: 24-35); Huppi et al (Mol. Cell. 2005 17: 1-10); Karagiannis et al (Cancer Biol. Ther. 2004 3: 1069-1074); and Shi et al (Trends Genet. 2003 19: 9-12).

Effective shRNA and siRNA molecules may be designed by known methods, see, e.g., Taxman et al (Biotechnol. 2006 6:7); McIntyre et al, (Biotechnol. 2006 6:1); Amarzguioui et al (FEBS Lett. 2005 579: 5974-81); Ito (FEBS Lett. 2005 579:5988-95); Bingbing et al (Nucl. Acids. Res. 2004 32:W130-W134) and Reynolds (Nat. Biotechnol. 2004 22: 326-30). Publicly available computer software for designing effective shRNA and siRNA molecules is also available from the Whitehead Institute for Biomedical Research, Genelink Inc. (Hawthorne, N.Y.), Invitrogen (BLOCK-iT RNAi Designer), The Wistar Institute (Gene specific siRNA selector), IDT (RNAi Design) and the Wadsworth Bioinformatics Center (siRNA Design) and Chang Bioscience, Inc. (Castro Valley, Calif.). In certain embodiments, the strands of an siRNA molecule may contain a symmetrical TT overhang at their 3′ ends (Elbashir 2001 Nature 411: 494-498 and Elbashir 2001 Nature 2001 411:494-8).

In one embodiment, an RNAi molecule may be a photocleavably linked double stranded molecule containing an RNA pol III promoter (e.g., a U6 or H1 promoter), a shRNA or siRNA sequence, and an RNA pol III terminator (e.g., 3 to 5 Ts), where the double stranded molecule is photocleavably linked to the subsbtrate. In other aspects of the invention, the double stranded molecule contains an RNA pol II promoter. Cleavage of this RNAi molecule releases a molecule that can be transfected into a cell to produce RNAi in the cell.

As mentioned above, a subject array contains RNAi molecules that are cleavably bound (i.e., bound indirectly or directly, covalently or non-covalently) to the surface of a substrate. In many embodiments, the capture agent is bound to the substrate via a cleavable linker. Cleavage of the cleavable linker may, in certain embodiments, result in breakage of a covalent bond that releases the RNA molecules.

Cleavable linkers that may be employed in the subject methods include electrophilically cleavable linkers, nucleophilically cleavable linkers, photocleavable linkers, metal cleavable linkers, electrolytically-cleavable, and linkers that are cleavable under reductive and oxidative conditions. Such linkers are described in great detail by Guillier et al (Chem. Rev. 2000 1000:2091-2157), which disclosure is incorporated by reference in its entirety.

In particular embodiments, a photocleavable linker (e.g., a uv-cleavable linker) may be employed. Suitable photocleavable linkers for use in a subject array include ortho-nitrobenzyl-based linkers, phenacyl linkers, alkoxybenzoin linkers, chromium arene complex linkers, NpSSMpact linkers and pivaloylglycol linkers, as described in Guillier et al, supra.

Of the photocleavable linkers, ortho-nitrobenzyl-based linkers are of particular interest since they can be made with a high yield and can be straightforwardly protected from cleavage-stimulating light (e.g., ultraviolet light, or “uv” light, i.e., light having a wavelength of 100 and 400 nm, e.g., UVA light (315-400 nm), UVB light (280-315 nm), UVC light (200-280 nm) or VUV light (100-200 nm)). Ortho-nitrobenzyl-based linkers contain an ortho-nitrobenzyl group. Ortho-nitrobenzyl-based linkers, suitable methods for their synthesis and their use in photocleavably linking biomolecules to a substrate are discussed in great detail by Guillier et al, supra and Olejnik et al (Methods in Enzymology 1998 291:135-154), and further described in U.S. Pat. No. 6,027,890; Olejnik et al (Proc. Natl. Acad Sci, 92:7590-94); Ogata et al. (Anal. Chem. 2002 74:4702-4708); Bai et al (Nucl. Acids Res. 2004 32:535-541); Zhao et al (Anal. Chem. 2002 74:4259-4268); and Sanford et al (Chem. Mater. 1998 10:1510-20).

In certain embodiments, a photocleavable RNAi molecule may be made using standard phosphoramidite chemistry. Methods for synthesis of photocleavable oligonucleotides (e.g., oligonucleotides that contain a photocleavable nucleotide or a photocleavable linker at an end of an oligonucleotide) are found in Olejnik et al (Proc. Natl. Acad. Sci. USA 1995, 92: 7590-4); Soukup et al (Bioconjugate Chemistry 1995 6: 135-138). Olejnik et al (Nucleic Acids Res. 1996 24: 361-6); Olejnik et al (Nucleic Acids Res. 1998 26: 3572-3576); Olejnik et al (Methods Enzymol. 1998 291: 135-54); Hahner (Biomol. Eng. 1999 16: 127-133); and Olejnik et al (Nucleic Acids Res. 1999 27: 4626-4631). In alternative embodiments, substrate may be derivatized to provide a photocleavable group, and an RNA oligonucleotide may be synthesized on that group. Such methods are described in, for example, Anderson et al, (Nucleosides Nucleotides & Nucleic acids 2003 22: 1403-1406).

In certain embodiments, an RNAi molecule can be tethered to a substrate using a suitable linking agent (e.g., a suitable ortho-nitrobenzyl-based linking agent) that possesses the following features, in order: a tag for linking to a substrate, a spacer moiety, a cleavable linker and a reactive group. The tag may be an affinity tag, e.g., a biotin group or the like, or a reactive moiety (e.g., a carboxy group, an amino group, a halo group, a tosylate group, a mesylate group, a reactive hydroxyl groups or metal oxide) that can react with suitable sites (e.g., alcohols, amino nucleophliles, thiol nucleophiles or silane groups) on the surface of a substrate to produce a covalent bond between the substrate and the linker. The spacer moiety may contain an unreactive alkyl chain, e.g., containing 3-12 carbon atoms (e.g., 5-aminocaproic acid) and the cleavable linker may be chosen as containing appropriate chemistry (see above). The reactive group may react with the oligonucleotide and forms a covalent bond therewith. The reactive group is selectively reactive with particular chemical groups in the capture agent. Suitable reactive groups include halogens (that are sulhydryl reactive), N-hydroxysuccinimide (NHS)-carbonate (that are amine-reactive) and N,N-diisopropyl-2-cyanoethyl phosphoramidite (that are hydroxyl-reactive), and several other reactive groups are known in the art and may be readily employed in the instant methods. In particular embodiment, a photocleavable ortho-nitrobenzyl NHS linking agent may be employed.

Exemplary linking agents that may be employed in the subject methods are described in Guillier et al, supra and Olejnik et al (Methods in Enzymology 1998 291:135-154), and further described in U.S. Pat. No. 6,027,890; Olejnik et al (Proc. Natl. Acad Sci, 92:7590-94); Ogata et al. (Anal. Chem. 2002 74:4702-4708); Bai et al (Nucl. Acids Res. 2004 32:535-541); Zhao et al (Anal. Chem. 2002 74:4259-4268); and Sanford et al (Chem. Mater. 1998 10:1510-20), and are purchasable from Ambergen (Boston, Mass.; NHS-PC-LC-Biotin), Link Technologies (Bellshill, Scotland), Fisher Scientific (Pittsburgh, Pa.; PIERCE EZ-LINK™ NHS-PC-LC-Biotin) and Calbiochem-Novabiochem Corp. (La Jolla, Calif.).

A cleavably bound RNAi molecule may be released from the substrate by exposing the substrate to light of 300-370 nm wavelength, e.g., 365 nm, at a suitable intensity, e.g., 1.2 mW/cm².

Arrays of RNAi molecules that contain RNAi molecules that are merely trapped at or near a surface of a substrate (e.g., because they were deposited upon the surface in conjunction with a trapping matrix, e.g., gelatin, agarose, acrylamide, fibrin, or the like) such that they can diffuse away upon addition of an aqueous media do not contain cleavably linked RNAi molecules. Such arrays are specifically excluded from this description. Binding between an RNAi molecule and the substrate of the array may be covalent, or may be non-covalent. If binding is non-covalent, the RNAi molecule may be bound to the substrate with an association constant K_{association} a of at least 10⁸ M⁻¹, 10⁹ M⁻¹,10¹⁰ M⁻¹, 10¹¹ M⁻¹, 10¹² M⁻¹, 10¹³ M⁻¹, 10¹⁴ M⁻¹ or 10¹⁵ M⁻¹, or more.

As would be recognized, RNAi molecules can be pre-made (e.g., synthesized by a machine or made by recombinant means) and then covalently bound to a cleavable linker that is affixed to the surface of a substrate. In alternative embodiments, RNAi molecules may be synthesized to contain a photocleavable linker, and then bound to a solid support. In other embodiments, an RNA molecule may be synthesized on a substrate in situ. In these embodiments, the substrate may be first derivatized to contain a photocleavable moiety upon which oligonucleotides are synthesized, or a photocleavable moiety may be added to a growing oligonucleotide during its synthesis.

In certain embodiments, the RNAi molecules of a subject array are oligonucleotides, e.g., oligoribonucleotides, and do not exceed about 200 nt in length, e.g., up to 100 or 150 nucleotides in length. A siRNA molecule present on a subject array or a double-stranded region of an shRNA molecule may be about 15 to 30 bases in length, e.g., about 15 to 29 bases, 20 to 29 bases or 21 bases to 22 bases, in length.

A subject array contains a plurality of RNAi molecules, where a plurality is meant at least 2, at least about 10, or at least about 25, and where the number of different RNAi molecules in the array may be at least about 500 or more, at least about 1000 or more, at least about 5000 or more, or at least about 10,000 or more etc., where each RNAi molecule is present in a single feature of the array.

A subject array may include a single RNAi agent for a given gene, such that each RNAi molecules on the array corresponds to a different gene or, in other embodiments, the subject array may contain multiple RNAi molecules for a given gene, where each of the multiple RNAi molecules is present at a distinct feature. The number of RNAi molecules that correspond to a given gene may range from about 2 to about 100 or more, such as from about 2 to about 50 or more, including from about 2 to about 25 or more. The RNAi molecules displayed on the array may be directed to genes of known or unknown function.

The total number of features on a substrate may vary depending on the number of different RNAi molecules to be explored or assayed, as well as the number of control features, calibrating features and the like, as may be desired. Generally, the pattern present on the surface of the support will comprise at least about 10 distinct features, at least about 200 distinct features, or at least about 500 distinct features, where the number of features can be as high as 50,000 or higher, but will usually not exceed about 25,000 distinct features. Each distinct RNAi molecule composition may be present in duplicate or more (e.g., at least 5 replicas) to provide an internal correlation of results.

The different RNAi molecules may be located in any position of the array. For example, in certain embodiments, the RNAi molecules may be randomly distributed across the array or grouped according to predetermined criteria, for example. In one embodiment adjacent features of an array may contain the different strands of a double stranded siRNA molecule.

The amount of RNAi molecule present in each feature will be sufficient to provide for adequate gene silencing in cells during the assay in which the array is employed. In certain embodiments, the feature will have an overall circular or square dimension, the diameter of which will range from about 10 μm to 5,000 μm, e.g., from about 20 μm to 1000 μm or about 50 μm to 500 μm.

A variety of substrates are suitable for use as a substrate of a subject array. A substrate may be flexible or rigid. By flexible is meant that the support is capable of being bent, folded or similarly manipulated without breakage. Examples of flexible solid supports include acrylamide, nylon, nitrocellulose, polypropylene, polyester films, such as polyethylene terephthalate, etc. In contrast, rigid supports do not readily bend, and include glass, fused silica, quartz; plastics, e.g. polytetrafluoroethylene, polypropylene, polystyrene, polycarbonate, and blends thereof, and the like; metals, e.g. gold, platinum, silver, and the like; etc.

In one embodiment, the substrate comprises a planar surface, and the RNAi molecules are spotted or synthesized in situ on the surface in an array. The features of the substrate can be any convenient shape, but will often be circular, elliptoid, oval or some other analogously curved shape. The local density of the spots on the solid surface can be at least about 500/cm² and usually at least about 1000/cm² but does not exceed about 10,000/cm², and usually does not exceed about 5000/cm², in many embodiments. The features to features distance (center to center) may from about 100 μm to about 1000 μm, however, in particular embodiments, the center to center distance of the features of a subject array may be the same as or twice the center to center distance of the wells of a multi-well plate. The features can be arranged in any convenient pattern across or over the surface of the support, such as in rows and columns so as to form a grid, in a circular pattern, and the like, where generally the pattern of features will be present in the form of a grid across the surface of the solid support.

The subject substrates can be prepared using any convenient means. One means of preparing the supports is to synthesize the RNAi molecules, and then deposit the pre-synthesized agents as a spot on the support surface. The RNAi molecules can be prepared using any convenient methodology, such as by chemical synthesis, in vitro transcription, and deposited onto the substrate. In another embodiment, the RNAi molecules are synthesized in situ, e.g., using an ink-jet printer or the like. Chemistry for the synthesis of oligonucleotides is described in, for example, in Caruthers, Science 230: 281-285, 1985; Itakura, et al., Ann. Rev. Biochem. 53: 323-356; and Hunkapillar, et al., Nature 310: 105-110, 1984, for example.

Methods

The above-described arrays may be employed in a method that includes a) contacting the array with: i) cells, and ii) an agent (e.g., light or a compound) that cleaves the cleavable linker to release the interfering RNA molecules from the substrate; b) introducing the interfering RNA molecules into the cells; and c) observing the cells. The agent employed in the method is generally compatible with (i.e., does not cause a detectable phenotypic effect, e.g., does not kill) the cells employed, in the method.

The cells and the agent may be contacted with the array simultaneously or in series. For example, a) the cells may be first mixed with the agent prior to their contact with the array, b) the cells may be contacted with the array prior to contacting the array with the agent, or the agent may be contacted with the array prior to contacting the array with the cells. In one embodiment, the cells are contacted with the array, and the array is illuminated with a light at an intensity and wavelength suitable to cause release of the RNAi molecules from the array.

In particular embodiments, the array is engaged, e.g., mated, with a multi-well plate such that the wells of the plate, when engaged with the array, form a sealed chamber containing the cells and one or more features (e.g., two, three or four features) of the array. If more than one feature are present in a sealed chamber, then those features may contain sense and antisense strands of a double-stranded siRNA molecule, or an shRNA molecule and a cleavable marker molecule that can be employed to monitor cleavage and transfection efficiency, for example.

In certain embodiments, the cells may be contained in an aqueous medium, and that medium may further contain transfection reagents that facilitate uptake of the released RNAi molecules into the cells. Such transfection reagents, e.g., EXGEN 500™, EFFECTINE™, JETSI™ and SUPERFEC™, are available from Qiagen (Carlsbad, Calif.) and MBI Fermentas (Hanover, Md.), and Qbiogene (Irvine, Calif.) among others. In other embodiments, the multi-well plate containing the cells may contain electrodes and may be employed in electroporation methods.

In practicing the subject methods, the subject arrays are contacted with multi-well plate containing in the wells thereof a cellular population made up of a plurality of distinct cells (e.g., a suspension of cells). In certain aspects, the population may be homogenous with respect to the nature of its constituent cells, such that all of the cells in the cell population contacted with the array are of the same type.

The type of cell that is contacted with the array may vary greatly, both in terms of species of origin and function. In many embodiments, the cells are from species that are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In certain embodiments, the cells will be human cells. Other types of cells include, but are not limited to: other animal cells, e.g., insects, invertebrates, and the like.

Cell types that can find use in the subject methods include stem and progenitor cells, e.g., embryonic stem cells, hematopoietic stem cells, mesenchymal stem cells, neural crest cells, etc., endothelial cells, muscle cells, myocardial, smooth and skeletal muscle cells, mesenchymal cells, epithelial cells; hematopoietic cells, such as lymphocytes, including T-cells, such as Th1 T cells, Th2 T cells, Th0 T cells, cytotoxic T cells; B cells, pre-B cells, etc.; monocytes; dendritic cells; neutrophils; and macrophages; natural killer cells; mast cells;, etc.; adipocytes, cells involved with particular organs, such as thymus, endocrine glands, pancreas, kidney, brain, such as neurons, glia, astrocytes, dendrocytes, etc. and genetically modified cells thereof. Hematopoietic cells may be associated with inflammatory processes, autoimmune diseases, etc., endothelial cells, smooth muscle cells, myocardial cells, etc. may be associated with cardiovascular diseases; almost any type of cell may be associated with neoplasias, such as sarcomas, carcinomas and lymphomas; liver diseases with hepatic cells; kidney diseases with kidney cells; etc.

The cells may also be transformed or neoplastic cells of different types, e.g. carcinomas of different cell origins, lymphomas of different cell types, etc. The American Type Culture Collection (Manassas, Va.) has collected and makes available over 4,000 cell lines from over 150 different species, over 950 cancer cell lines including 700 human cancer cell lines. The National Cancer Institute has compiled clinical, biochemical and molecular data from a large panel of human tumor cell lines, these are available from ATCC or the NCl (Phelps et al. (1996) Journal of Cellular Biochemistry Supplement 24:32-91). Included are different cell lines derived spontaneously, or selected for desired growth or response characteristics from an individual cell line; and may include multiple cell lines derived from a similar tumor type but from distinct patients or sites.

Cells may be non-adherent, e.g., blood cells including monocytes, T cells, B-cells; tumor cells, etc.; or adherent cells, e.g., epithelial cells, endothelial cells, neural cells, etc. In order to employ adherent cells, the cells are typically dissociated from the substrate that they are adhered to, and from other cells, in a manner that maintains their viability. Methods of dissociating cells are known in the art, including protease digestion, etc. In certain embodiments, the dissociation methods use enzyme-free dissociation media.

The cells are contacted with the RNAi array under conditions sufficient for the cells to be transfected with the RNAi molecules upon their release from the array. As such, a multi-well plate containing cells is placed over the substrate surface and engaged thereto, and the multi-well plate and substrate are inverted such that the cells become in contact with the array. After the RNAi molecules have been released, the multi-well plate and substrate may be inverted again so that the cells and the RNAi molecules are in the wells of the plate and the RNAi molecules may be introduced into the cells. In other words, the cells are contacted with the array under transfecting conditions in which released RNAi molecules are introduced into the cells.

Following contact or plating of the cells onto the array surface, the resultant cells are maintained on the surface under suitable conditions and for a sufficient period of time for the cells to be transfected by the various RNAi agents. In certain embodiments, the resultant cells are maintained at a temperature ranging from about 20° C. to about 40° C., such as from about 25° C. to about 39° C., for a period of time for one or more, e.g., one to five, including one to three, e.g., two cell cycles to occur.

After sufficient time has elapsed, the cells are assessed for transfection (entry of the RNAi molecules into the cells) and/or effect of the introduced RNAi molecules on transfected cells, e.g., by using known methods. In certain embodiments, the cells are assayed or evaluated for any phenotypic variation from the wild-type phenotype.

Various cellular outputs may be assessed to determine the response of the cells to the input RNAi, including calcium flux, BrdU incorporation, expression of an endogenous or a transgene reporter, metabolic reporters, electrical activity (e.g. via voltage-sensitive dyes), release of cellular products, cell motility, size, shape, viability and binding, etc. Generally the analysis provides for well-specific determination, i.e., the cells that are present in a well are analyzed for phenotype that correlates with the RNAi molecule input into the cells.

The phenotype of a cell in response to an RNAi may be detected by detecting an observable characteristic of a cell, where characteristics of interest include cell morphology, growth, viability, expression of genes of interest, interaction with other cells, and include changes in quantifiable parameters and parameters that can be accurately measured.

A parameter can be, for example, the amount of any cell component (including its presence or absence) or the localization of any cell component (i.e., where a cell component is localized). For example, a parameter can be, in certain embodiments, a cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. Parameters may provide a quantitative readout, or in some instances, a semi-quantitative or qualitative result. Readouts may include a single determined value, or may include mean, median value or the variance, etc. Variability is expected and a range of values for each of the set of test parameters will be obtained using standard statistical methods with a common statistical method used to provide single values.

Parameters of interest include detection of cytoplasmic, cell surface or secreted biomolecules, biopolymers, e.g. polypeptides, polysaccharides, polynucleotides, lipids, etc. Cell surface and secreted molecules are a useful parameter type as these mediate cell communication and cell effector responses and can be readily assayed.

As such, a variety of methods can be used to detect the consequence of uptake of the RNAi molecule. In a general sense, the assay provides the means for determining if the RNAi agent is able to confer a change in the phenotype of the cell relative to the same cell but which lacks the RNAi molecule. Such changes can be detected on a gross cellular level, such as by changes in cell morphology (membrane ruffling, rate of mitosis, rate of cell death, mechanism of cell death, dye uptake, and the like). In other embodiments, the changes to the cell's phenotype, if any, are detected by more focused means, such as the detection of the level of a particular protein (such as a selectable or detectable marker), or level of mRNA or second messenger, to name but a few. Changes in the cell's phenotype can be determined by assaying reporter genes (beta-galactosidase, green fluorescent protein, beta-lactamase, luciferase, chloramphenicol acetyl transferase), assaying enzymes, using immunoassays, staining with dyes (e.g. DAPI, calcofluor), assaying electrical changes, characterizing changes in cell shape, examining changes in protein conformation, and counting cell number. Other changes of interest could be detected by methods such as chemical assays, light microscopy, scanning electron microscopy, transmission electron microscopy, atomic force microscopy, confocal microscopy, image reconstruction microscopy, scanners, autoradiography, light scattering, light absorbance, NMR, PET, patch clamping, calorimetry, mass spectrometry, surface plasmon resonance, time resolved fluorescence. Data could be collected at single or multiple time points and analyzed by the appropriate software.

Detection of a phenotypic change in cells contacted with a given RNAi molecule can then used to determine the activity of the RNAi molecule with respect to expression of a gene to which it corresponds, as described above. Specifically, a change in a cell phenotype as compared to a control observed in cells contacted with a given RNAi molecule means that that RNAi molecule has activity in modifying, and typically reducing, expression of the gene to which it corresponds.

Also provided is a system for performing the above methods. In certain embodiments, the system comprises at least a subject array and a multi-well plate, e.g., a multi-well plate containing, for example, 24, 96, 384, 1536 wells. The format of the multi-well plate and/or the array may be adapted so that each of the wells of the multi-well plate aligns with a feature (or a group of features) when the plate is operably engaged with the array. In other words, in certain embodiments, the array substrate and the multi-well plate may be adapted to fit together such that a series of sealed chambers are formed that each contain one or more features (e.g., a single feature or two, three or four features). The multi-well plate and/or the array may contain a sealing element, e.g., a gasket that permits the sealing of each of the chambers. This sealing element, if it is in the multi-well plate, may contact an inter-feature area of the array. The multi-well plate and/or the array may contain reference marks or alignment elements, e.g., pins and holes to facilitate accurate alignment of the plate to the array. In certain embodiments, the multi-well plate may be adapted for culturing cells.

Kits

Also provided are reagents and kits thereof for practicing one or more of the above-described methods. The subject reagents and kits thereof may vary greatly. Typically, the kits at least include a subject array as described above and, in certain embodiments, a multi-well plate adapted for mating with the array, as described above.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

Utility

The subject arrays and methods for using the same, as described above, find use in a number of different applications. One representative method of using the subject arrays is to determine the activity of two or more different RNAi molecules with respect to one or more genes, and specifically the expression of one or more genes, of a given type of cell. In these embodiments, the impact of two or more different RNAi molecules on the expression of one or more different genes in a given cell is determined at substantially the same, if not the same, time, e.g., as may be found in a high throughput format.

The above-described arrays and methods for using the same also find use in loss-of-function genetic assays, particularly in high-throughput formats of such assays. As such, the subject RNAi arrays can be used to assess the loss-of-function of a particular gene or genes. In such loss-of-function applications, an array of a plurality of different RNAi molecules directed to one or more different genes is contacted with a cell suspension, as described above, under transfection conditions. Following transfection, the cells contacted with the RNAi molecules are evaluated for phenotypic change, as described above. The location of cells exhibiting phenotypic variation of interest is then employed to determine the identity of the RNAi molecules that transfected the cells of interest and caused the phenotypic change of interest. Identification of the RNAi molecules is then used to determine the identity of the gene whose expression has been inhibited or reduced, resulting in the observed phenotype of interest. In this way, the function of the gene is extrapolated. In other words, the gene may be annotated with respect to it function.

Because arrays of RNAi molecules are contacted with a plurality of cells and the resultant transfected cells are evaluated at the substantially the same, if not the same, time, the subject arrays and methods for using the same are particularly suited for use in high throughput loss of function genomic assays.

Representative utilities are also described in U.S. Application Publication No. 20020006664 (the disclosure of which is herein incorporated by reference).

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A nucleic acid array comprising:
a) a substrate; and
b) an array of features on a surface of said substrate, where said features comprise interfering RNA molecules that are linked to said surface of said substrate by a cleavable linker.

2. The nucleic acid array of claim 1, wherein said interfering RNA molecules comprise short interfering RNA molecules.

3. The nucleic acid array of claim 1, wherein said interfering RNA molecules comprise short hairpin interfering RNA molecules.

4. The nucleic acid array of claim 1, wherein said interfering RNA molecules are linked to said surface via a photocleavable linker.

5. The nucleic acid array of claim 1, wherein said interfering RNA molecules are linked to said surface via a chemically-cleavable linker.

6. The nucleic acid array of claim 1, wherein said interfering RNA molecules are covalently linked to said surface of said substrate.

7. A method comprising:
a) contacting a nucleic acid array of claim 1 with:
i) cells, and
ii) an agent that cleaves said cleavable linker to release said interfering RNA molecules from said substrate;
b) introducing said interfering RNA molecules into said cells; and
c) observing said cells.

8. The method of claim 7, wherein said agent is light.

9. The method of claim 7, wherein said agent is a compound.

10. The method of claim 7, wherein said cells and said agent are contacted with said array simultaneously.

11. The method of claim 7, wherein said cells and said agent are contacted with said array in series.

12. The method of claim 7, wherein said observing is observing a phenotype of said cells.

13. The method of claim 7, wherein said observing includes detecting a reporter protein.

14. The method of claim 7, further comprising comparing said cells to control cells.

15. The method of claim 7, wherein said contacting includes operably engaging said nucleic acid array with a multi-well plate comprising said cells.

16. A system comprising:
an array of claim 1; and
a multi-well plate of cells;
wherein said array and multiwell plate are adapted for engaging to each other such that said cells come into contact with one or more pre-determined features of said array.

17. The system of claim 16, wherein said array and said multi-well plate comprise alignment elements that provide alignment of said array and said multi-well plate as they are being engaged.

18. The system of claim 16, wherein said alignment elements include reference marks.

19. The system of claim 16, wherein said array and said multi-well plate, once engaged, produces a plurality of sealed chambers.

20. A kit comprising
an array of claim 1;
a multi-well plate; and
a transfection reagent;
wherein said array and multiwell plate are adapted for engaging to each other such that said array and said multiwell plate, when engaged, form a plurality of sealed reaction chambers.
